(19) United States
(12) Patent Application Publication
(10) Pub. No.: US 2005/0136393 A1
(43) Pub. Date: Jun. 23, 2005

(54) Oligodoxynucleotide (ODN) libraries, their use in screening for antibacterial agents, and catalytic ODN sequence for use as an antibacterial agent

(75) Inventors: Yin Chen, Pearland, TX (US); Xin Xing Tan, Manvel, TX (US)

Correspondence Address: Wisner & Associates, Suite 400, 1177 West Loop South, Houston, TX, 77027-9012, US

(73) Assignee: CytoGenix, Inc., Houston, TX (US)

(21) Appl. No.: 10/743,956

(22) Filed: Dec. 23, 2003

(51) Int. Cl. C12Q 1/00; C12Q 1/68; C12N 9/22; C12N 15/85

(52) U.S. Cl. 435/4; 435/455; 435/199; 435/325

(57) ABSTRACT

A selectively inducible, single-stranded DNA (ssDNA) expression library, a method for constructing a ssDNA expression library, a method for screening ssDNA using the expression library, and a method for identifying ssDNA molecules that alter expression of bacterial gene(s) related to cell growth and toxin production and secretion. The screening library is used to, among other things, identify ODNs effective in stopping bacterial growth, killing bacteria or preventing bacteria from synthesizing and secreting their toxins, and/or to discover ODNs effective in eukaryotic (e.g., mammalian) cells for targeted alteration of gene function. The library is also useful for identifying ssDNAs or ODNs that are used as therapeutic antibacterial reagents, for identifying essential bacterial genes that can serve as targets for antibiotic discovery, and for providing a method for treatment of bacterial infections.

### BACKGROUND OF THE INVENTION

Oligonucleotide-mediated intervention (OMI) technology provides a powerful tool for altering the activity of any gene of known sequence. The ability to produce single strands of DNA (ssDNA) of any sequence and length in selected cells enables targeted alteration of gene expression at the genomic level using triplex-forming oligonucleotides (TFOs) for targeted gene expression, at the messenger RNA (mRNA) level using antisense and DNA enzyme oligos, and at the protein level using ssDNA as aptamers (Chen, Y. 2002, Expert Opin. Biol. Ther. 2(7) 735-740).

Antisense, DNA enzyme, triplex, and aptamer technologies provide an efficient alternative to more difficult methods such as creating gene knockout in cells and organisms. Antisense oligonucleotides (ODNs) block gene expression by Watson-Crick base pairing between an ODN and its target mRNA (Crooke, S. T. 1999, Biochim. Biophys. Acta 1489:31-44). Antisense ODNs have been used to effectively inhibit gene expression in eukaryotic cells and have been used to validate gene targets. There is one antisense ODN-based product in the market and a number of others in advanced clinical trials (Uhlman, E., 2001, Expert Opinion on Biological Therapy, 1:319-328). However, antisense technology is not used extensively in prokaryotic systems. Prokaryotic cells have themselves developed endogenous antisense mechanisms for gene regulation (Simons & Kleckner, 1988, Annu. Rev. Genet., 22, 567-600). Earlier results indicated that gene expression in bacteria may be accessible to inhibition by modified ODNs (Jayayaraman, et al., 1981, PNAS, 78:1537-1541; Gasparro, F. P., et al., 1991, Antisense Res Dev., 1:117-140). Others reported that peptide nucleic acid (PNA) can inhibit gene expression in bacteria. (Good & Nielsen, 1998, Nature Biotechnology, 16:355-358). PNA, a DNA mimic in which the nucleotide bases are attached to a pseudopeptide backbone, hybridizes with complementary DNA, RNA, or PNA oligomers through Watson-Crick base pairing and helix formation.

One major parameter determining efficacy of any OMI strategy is target site accessibility. The lack of effectiveness of antisense, or other ODNs, may largely be a result of selecting inaccessible sites in the target. Base composition can undoubtedly affect heteroduplex formation, but it does not appear to be the primary factor. There is now convincing evidence that binding of complementary ODNs is mainly determined by the secondary and tertiary structures of RNA molecules (Frauendorf A., et al., Bioorg. Med. Chem. Lett., 1996, 4:1019-1024).

Various approaches to identifying the accessible sites on target mRNAs in relation to antisense and/or DNA enzyme design have been developed. Conventionally, a linear shot-gun approach has been used to select antisense ODNs. Several oligonucleotides, targeted to various regions of an mRNA, are synthesized individually and their antisense, DNA enzymatic or other activity (or binding affinity to the target sites) measured. However, only 2-5% of ODNs are generally found to be good antisense reagents.

In an attempt to introduce rationality and efficiency into efforts to identify active OMI reagents, researchers also use computer programs. For instance, the secondary structure of target RNA is predicted using an RNA folding program such as mfold (M. Zuker, 1989, Science, 244, 48-32). Antisense ODNs are designed to bind to regions that are predicted to be free from intramolecular base pairing. However, energy-based prediction methods of RNA structure are largely inadequate for designing antisense reagents and success using this approach has been limited.

Evidence that ribonuclease H (RNase H) is involved in antisense-mediated effects has led to the development of several procedures that make use of this enzyme to identify accessible binding sites in mRNAs in vitro. RNase H is an endoribonuclease that specifically hydrolyzes phosphodiester bonds of RNA in DNA:RNA hybrids. For one such procedure, RNase H is used in combination with a random ODN library comprising a complete set of all possible ODNs of a defined length to identify accessible sites in mRNA. For instance, for a length N, there are N⁴ different possible ODNs that would comprise the ODN library set such that there are approximately 2.56×10⁶ molecules for a 40-mer ODN. Component ODNs of the library that are complementary to accessible sites on the target RNA produce hybrids with RNA that are identified as RNase H cleavage sites by gel electrophoresis. While many of the possible ODNs in the library set are of no interest (e.g., an ODN such as AAAA . . . AAAA), the library set members are tested to see which, if any, produces a down-regulating effect on a specific target mRNA. Controlled gene expression systems such as the tetracycline regulatory system in prokaryotic cells allow selective gene down- or up-regulation and thereby supply information on the gene product.

Hammerhead and hairpin ribozymes are catalytic RNA molecules that bind and enzymatically cleave defined RNA targets and have been used successfully to knock down gene expression of viral and cellular targets (see James, H. A. & Gibson, I., Blood, 91:371-382, 1998). A method has been developed to identify accessible sites on the ICP4 mRNAs for antisense-mediated gene inhibition using a hammerhead ribozyme library that allows expression of the library components in mammalian cells (Pierce & Ruffner, 1998, Nucleic Acid Research, 26:5093-5101). ICP4 is an essential transcriptional activator of the Herpes simplex virus (HSV). Although hammerhead ribozymes can efficiently cleave specific mRNA targets, clinical application of this method is limited because of instability caused by RNase degradation in vivo.

The ability to identify a gene or gene family that is responsible for a particular phenotype is crucial to the understanding of any biological mechanism of disease. Ribozyme libraries can be used not only to identify accessible sites on target mRNA, but also genes that are directly involved in producing a particular phenotype. Researchers from Immusol, Inc. constructed a hairpin ribozyme library that was delivered to mammalian cells either with plasmid or retroviral vectors (Welch, P. J., et al., Genomics, 66, 274-283, 2000, Li, Q., et al., Nucleic Acid Research, 28:2605-2612, 2000, Kruger, M., et al., PNAS, 97:8566-8571, 2000, Beger, C., et al., PNAS, 98:130-135, 2001). By knocking-down or knocking-out gene expression using a ribozyme library, they were able to identify novel gene or new functions of known genes such as 1) the human homologue of the Drosophila gene ppan, involved in mammalian cell growth control 2) telomerase reverse transcriptase (mTERT), a suppressor of cell transformation; 3) eukaryotic translation initiation factors, eIF2Bγ and eIF2γ, as cofactors of hepatitis C virus internal ribosome entry site-mediated translation; and 4) transcriptional regulator Id4 as a regulator of BRCA1 gene expression. However, similar to hammerhead ribozymes, hairpin ribozymes have limited stability in vivo.

Ji, et al. constructed a library of small staphylococcal DNA fragments (200 to 800 bp) derived by shearing genomic DNA (Ji, et al., 2001, Science, 293:2266-2269). By transforming the library into Staphylococcus aureus, random antisense RNA molecules were generated. Using this approach, critical genes were identified that could serve as targets for antibiotic discovery. A similar approach was used by Forsyth, et al. in S. aureus (Forsyth, et al., 2002, Molecular Microbiology, 43:1387-1400). However, this approach can only be used for the identification of essential genes since antisense RNA with the size between 200-800 bp is not useful for therapeutic purposes because of 1) the instability of RNA molecules; 2) the difficulty of synthesizing RNA molecules with the size of 200-800 bp; and 3) the problem of delivering RNA to appropriate cells.

Traditional antibiotics are low-molecular-weight compounds that either kill (bactericidal) or inhibit (bacteriostatic) the growth of bacteria. Most are produced by microorganisms, especially Streptomyces spp. and fungi. Antibiotics are directed against targets that are preferably specific to bacteria, minimizing their potential toxicity to humans. Specific targets include inhibitors of cell wall biosynthesis, aromatic amino acid biosynthesis, cell division, two component signal transduction, fatty acid biosynthesis, isopreniod biosynthesis and tRNA synthesis. For example: 1) Penicillin blocks the final step of cell wall synthesis by binding covalently to the active site of the transpepetidase enzyme; 2) Kanamycin inhibits protein synthesis by interacting with bacterial ribosomal 30S RNA; 3) Rifampicin binds to the s subunit of bacterial RNA polymerase, the enzyme required to transcribe mRNA from the bacteria DNA; 4) Trimethoprim, which is a bacterial dihydrofolate reductase inhibitor while leaving the mammalian enzyme virtually unaffected; and 5) Ciprofloxacin, which inhibits bacterial toposiomerase II or DNA gyrase, the enzyme that controls the supercoiling or folding of the bacterial chromosome DNA within the cells. Inhibitors in categories 4) and 5) are not traditional antibiotics since they are completely synthetic compounds.

In recent years, there has been a rapid emergence of antibiotic resistance to many common bacterial pathogens such as S. aureus, Streptococcus pneumoniae and Enterococcus faecalis (Nicolaou, K. C. & Boddy, C. N. C., 2001, Scientific American, p.56-61). Methicillin-resistant S. aureus (MRSA), penicillin-resistant S. pneumococcus and vancomycin-resistant E. faecalis (VRE) are now common pathogens that are difficult to treat effectively (Pfaller, M. A., et al., 1998, Antimicrobiol Agents and Chemotherapy, 42:1762-1770; Jones, R. N., et al., 1999, Microbiology and Infections Disease, 33:101-112). Also alarming is the emergence of multi-drug resistance pathogens (Swartz, M. N., 1994, PNAS, 91:2420-2427; Baquero, F., 1997, J. Antimicrobial Chemotherapy, 39:1-6). Until recently, the principal approach of the pharmaceutical industry has been to seek incremental improvements in existing drugs. Although these approaches have made a significant contribution to combating bacterial infections, they are having difficulty meeting the increasing needs of the medical community. Health care workers are finding that more and more of the weapons in the arsenal of antibiotics are becoming useless. Infectious diseases such as tuberculosis, meningitis and pneumonia, that would have been easily treated with antibiotics at one time, are no longer so readily thwarted.

There is, therefore, an emergent demand for the discovery and development of new classes of antibiotics to add to the current arsenal. Recent advances in DNA sequencing technology have made it possible to elucidate the entire genome sequences of pathogenic bacteria. Genomic sequencing reveals all of the information in bacteria related to potential targets by antibiotics and therefore provides a more rational target-based approach to develop new antibiotics.

The use of a screening library to identify ODNs effective in stopping bacterial growth, killing bacteria or preventing bacteria from synthesizing and secreting their toxins is the focus of the present invention. Use of the screening library to discover ODNs effective in eukaryotic (e.g., mammalian) cells for targeted alteration of gene function is a logical application.

It is, therefore, an object of the present invention to provide a method for identifying ssDNAs or ODNs such as triplex forming oligos (TFOs), antisense oligos, DNA enzymes, or aptamers that are used as therapeutic antibacterial reagents.

An additional object of the present invention is to provide a method for identifying essential bacteria genes that can serve as targets for antibiotic discovery.

An additional object of the present invention is the provision of a method for treating bacterial infections.

An additional object of present invention is to provide a method for regulating gene expression in eukaryotic cells in a controlled manner using a selectively-inducible expression vector such as the tetracycline system.

An additional object of present invention is to provide a method for regulating gene expression in eukaryotic cells in a controlled manner using an inducible vector such as the tetracycline system.

Yet another object of the present invention is to provide a novel antibacterial agent identified by practicing the method of the present invention.

### SUMMARY OF THE INVENTION

The present invention is a selectively-inducible single-stranded DNA (ssDNA) expression library, a method for constructing the ssDNA expression library, a method for screening ssDNA expression library, and a method for identifying ssDNA molecules that switch bacterial gene(s) related to cell growth, replication, and/or toxin production and secretion on or off.

The method comprises a method for constructing a set of randomly ordered, fixed length oligodeoxynucleotide (ODN) strands and sub-cloning these ODNs into expression vectors constituted so that, when transformed into cells that are subsequently exposed to certain chemical environments, the cell reacts by expressing the individual ODN sequence programmed into the expression vector. Cells containing the instructions for an individual ODN are grown into colonies and each of the colonies is divided into control and experimental sets. When an experimental colony is exposed to the external chemical inducing the production of an ODN, the ODN is expressed and putatively alters cellular gene function, for instance, protein production, producing a different cell phenotype. If the phenotypic expression represents a desired end result, the control colony is treated to extract the DNA to determine the exact nucleotide sequence of the ODN that produced the phenotype in question.

This method is used to identify ODNs that, for instance, kill bacterial cells, thereby making it possible to identify new antibiotics against pathogenic bacteria and provide methods for identifying essential bacterial genes that can serve as additional targets for discovery of new antibiotics. The same methods and screening library is utilized to identify ssDNA molecules that switch bacteria gene(s) related to cell growth and/or replication and toxin production and secretion on and/or off.

The present invention also includes a novel ssDNA enzyme having a catalytic sequence flanked by random targeting sequences that is effective in killing bacterial cells into which the sequence is introduced and/or in which the sequence is synthesized in vivo.

### BRIEF DESCRIPTION OF THE DRAWINGS

FIG. 1 is a schematic representation of a preferred embodiment of the expression vector pssXG constructed in accordance with the teachings of the present invention.

FIG. 2 is a schematic representation of a preferred embodiment of the FtsZ gene targeted DNA enzyme expression vector, pssXGb(FtsZ-DZ), constructed in accordance with the teachings of the present invention.

FIG. 3 shows the results of an activity assay for expression of HIS-tagged reverse transcriptase (RT) induced by tetracycline in E. coli. Bacterial cells carrying pssXGb(FtsZ-DZ) vector were grown at 37° C. until OD 280 value of 0.5 and then 200 ng/ml of aTc, a derivative of tetracycline, was added to the cells and incubated for another 1-2 hours. Cell lysates were used for the assay, conducted in accordance with Silver, et al. (1993, Nucleic Acids Res. 21: 3593-3594). Lane 1: without tetracycline induction; Lane 2: one hour aTc induction; Lane 3: two hour aTc induction. PCR amplification product is marked by an arrow.

FIG. 4 shows the results of a western blot assay for expression of HIS-tagged reverse transcriptase (RT) induced by tetracycline in E. coli. Bacterial cells carrying pssXGb(FtsZ-DZ) vector were grown at 37° C. in the presence of 0, 100, or 200 ng/ml aTc for 1 hour (lanes 1-3), 2 hours (lanes 4-6); or 3 hours (lanes 7-9). Cell lysates we used for the assay and the RT band is marked with an arrow.

FIGS. 5A and 5B show the design of the FtsZ mRNA-cleaving DNA enzyme and its targeting sequence, respectively.

FIG. 6 shows the results of an in vitro cleavage of FtsZ RNA. Lane 1 is a control reaction incubated for 2 hrs in the absence of DNA enzyme. S: substrate; P1 (857nt) and P2 (368nt): DNA enzyme digestion products.

FIG. 7 shows the results of DNA enzyme-mediated repression of ftsZ gene expression: A. bacterial cells carrying the pssXGb(FtsZ-DZ) vector; B. bacterial cells carrying the control pssXGa vector.

FIG. 8A shows the results of inhibition of cell proliferation by in vivo expressed DNA enzyme.

FIG. 8B shows the morphology of bacterial cells carrying pssXGb(FtsZ-DZ): A. bacterial cells carrying pssXGb(FtsZ-DZ), without aTc; B. bacterial cells carrying pssXGb(FtsZ-DZ), with 800 ng/ml aTc; C. bacterial cells carrying vector pssXGa, without aTc; and D. bacterial cells carrying pssXGa, with 800 ng/ml.

FIG. 9 shows the result of library screening. Colonies were grown in duplicate in LB plates with or without aTc. Positive clone (containing the pssXGb vector including the sequence CYGX080103) is marked by an arrow.

FIG. 10 shows the results of reconfirming the bacterial inhibitory effect of the positive clone (CYGX080103) from library screening. The CYGX080103 expression plasmid and the plasmid without ODN insert as negative control were transformed into DH5αPro, and plated onto LB media with or without 200 ng/ml aTc. FIG. 10A shows that DH5αPro carrying the CYGX080103 expression plasmid grows normally on the media without aTc, but not on the media with aTc. DH5αPro carrying the plasmid without the ODN insert grows on both media (FIG. 10B).

FIG. 11 shows the results of transformation of ODN (CYGX080103) expression vector into E. coli Xl10-gold (kan) cells. The transformants were plated on LB media with chloramphenicol, and incubated at 37° C. O/N. No XL10-gold(kan) cells carrying CYGX080103 expression plasmid grew on the LB media, but the XL10-gold(kan) cells carrying the plasmid without the ODN insert grew normally.

### DETAILED DESCRIPTION OF THE PREFERRED EMBODIMENT

Construction of Tetracycline-Inducible Prokaryotic ssDNA Expression Vector.

PCR amplification was carried out using the plasmid pssXE, described in International Application No. PCT/US00/27381, which application is hereby incorporated into this specification in its entirety by this specific reference, as the template. DNA primers used in the PCR reaction, 5′NheIPvuIATG

[Seq. ID No. 1]

5′-CTAGCTAGCTAGCGATCGATGGGACCAATGGGGCAG-3′
and 3′KpnI

[Seq. ID No. 2]

5′-CGGGGTACCAGTATTCCCTGGTC-3′

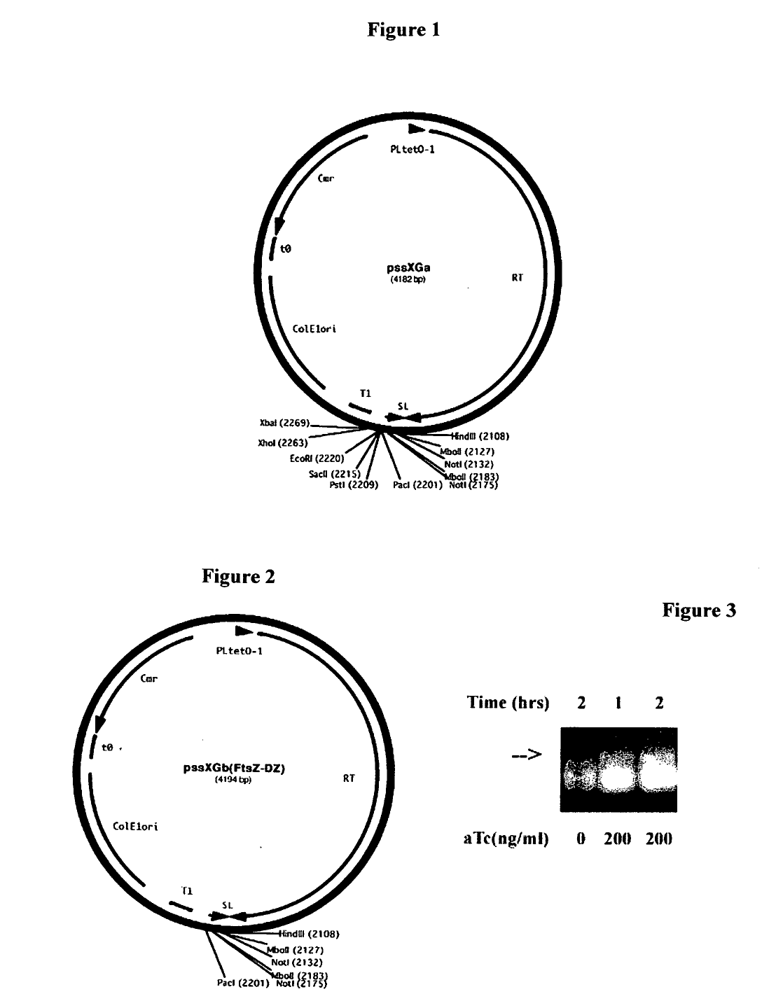

were synthesized by Integrated DNA Technologies (Coralville, Iowa). The PCR amplified DNA fragment was double-digested with NheI and Kpnl and then subcloned into the pssXE vector that was double-digested with the same enzymes. The replacement removes the sequence before the translation starting site (ATG), which is unnecessary for prokaryotic gene expression, while creating a new restriction enzyme site, PvuI. The newly created construct was digested with PvuI and XbaI. The PvuI-XbaI fragment contains all the essential elements for ssDNA production, including: 1) Mouse Moloney leukemia viral reverse transcriptase (MoMuLV RT) gene coding for a truncated but fully active RT (Tanase & Goff, PNAS, 2000, 85:1777-1781); 2) primer binding site (PBS) along with some flanking regions of the promoter that are essential for the reverse transcription initiation by MoMuLV RT (Shinnick, et al., Nature, 1981, 293:543-548); and 3) stem-loop structure designed for the termination of the reverse transcription reaction all as described in the above-incorporated International Application No. PCT/US00/27381. This DNA fragment was subcloned into the pPROTet.E 233 vector (BD Bioscience, Palo Alto, Calif.) and the newly created construct was designated as pssXGa, shown in FIG. 1. However, the sequence of bacteria tRNAPro is different from mammalian tRNAPro, which was designed to bind with the PBS in mammalian cells. Because bacterial tRNAVal can be utilized as primer for RT, a new PBS was designed to replace the PBS used in the vector pssXE that is used for mammalian cells. The sequence of the novel PBS is
5′-TGGTGCGTCCGAG-3′ [Seq. ID No. 3].

To construct a vector expressing the desired DNA enzyme and replace the original primer binding site (PBS) in the expression cassette of pssXE(CMV), the ODNs of sequence

[Seq. ID No. 4]

5′-dTAACTGGATGATCGTTGTAGCTAGCCTTCGAAACTTGGTGGTGCGT
CCGAGTGGACCGGGAGACCCCTGCTCGAGT-3′
and

[Seq. ID No. 5]

5′-CTAGACTCGAGCAGGGGTCTCCCGGTCCACTCGGACGCACCACCAAG
TTTCGAAGGCTAGCTACAACGATCATCCAGTTAAT-3′

were annealed to produce a synthetic duplex with 5′PacI and 3′XbaI cohesive ends and ligated into the PacI and XbaI sites of pssXGa. The resulting vector was designated pssXGb(FtsZ-DZ) and is shown in FIG. 2.

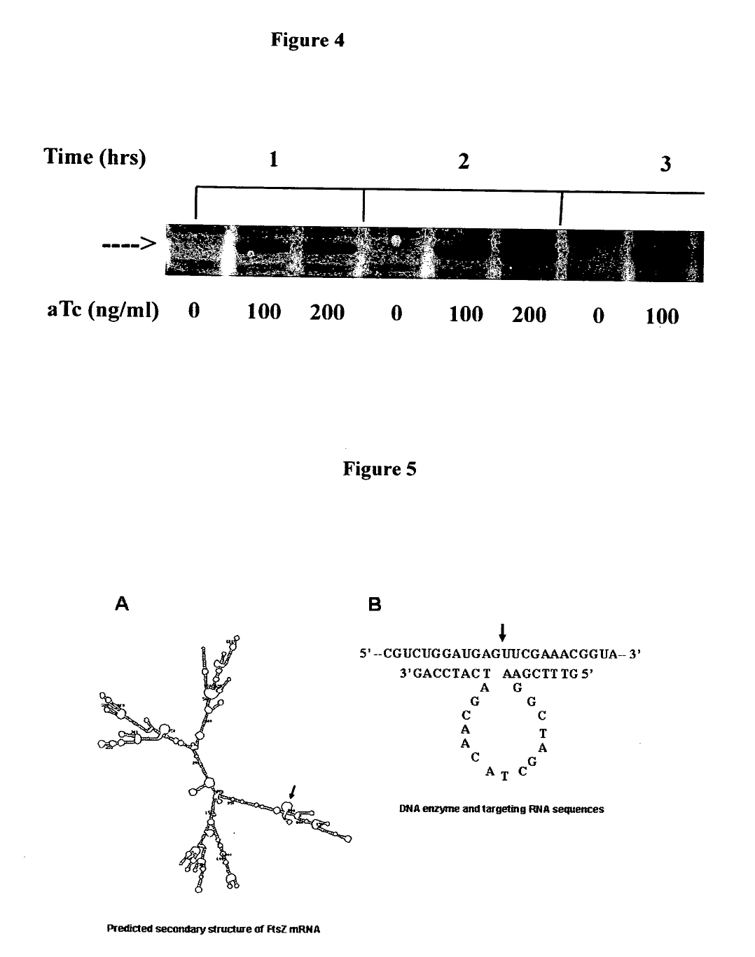

pPROTet.E233 is a tetracycline-inducible bacterial expression vector expressing fusion protein with 6xHN. It utilizes a novel promoter, P_{Ltet}O1, which is tightly repressed by the highly specific Tet repressor protein and induced in response to anhydrotetracycline (aTc), allowing control of induction over a wide range (anhydrotetracycline is a derivative of tetracycline that acts as a more potent inducer of PROTet.E Systems). The pssXG vector was transformed into the bacteria strain, DH5αPro (BD Bioscience, Palo Alto, Calif.) in the presence of 34 μg/ml choloramphenicol (Cm) and 50 μg/ml spectinomycin (spec). Spectinomycin is used to select for DH5αPro cells that carry transcription units encoding TetR (Lutz & Bujard, Nucleic Acids Res., 1997, 25:1203-1210). The DH5αPro cells express defined amounts of the Tet repressors. Cell lysates were prepared using B-PER II Bacterial Protein Extraction Reagent (Pierce, Rockford, Ill.) according to the manufacturer's instruction. Using the cell lysates, the expression of reverse transcriptase (RT) was confirmed by RT activity assay using cell lysates according to Silver, et al. (Nucleic Acids Res., 1993, 21:3593-3594) as shown in FIG. 3 and Western blotting using antibody against 6xHN (BD Bioscience, Palo Alto, Calif.) as shown in FIG. 4.

Inhibition of Bacterial Growth By DNA Enzyme Targeted To FtsZ

Cell division is critical for bacterial survival; bacteria such as Escherichia coli normally divide by binary fission, producing two daughter cells of equal size, each containing a nucleoid. FtsZ is an essential gene for bacterial division and viability. The division process starts with the localization of FtsZ directed to the center of the mother cell and formation of a septal structure, the Z ring. Other essential cell division proteins are then recruited to the Z ring. Deletion and mutation of the ftsZ gene blocks cell division at an early stage, showing promise as a target for developing a new antibiotic agent.

Because of their ability to bind and cleave any target RNA at purine/pyrimidine junctions, DNA enzymes are capable of interfering with gene expression as described in the above-incorporated International Application No. PCT/US00/27381. 10-23 DNA enzyme cleavage sites are plentiful in most biological substrates and thus provide a host of opportunities to achieve maximum cleavage efficiency. Based on the predicted secondary structure of FtsZ mRNA, a 10-23 DNA enzyme targeted against a GU site at position 880 was designed. The sequence of this 31 nt DNA enzyme is
5′-GTTTCGAAGGCTAGCTACAACGATCATCCAG-3′ [Seq. ID No. 6]
   with the predicted free energy 21.3 kcal/mol (FIG. 5). It will be apparent from FIG. 5 that the enzyme of the present invention is comprised of a 15 nucleotide catalytic domain flanked by random target-binding domains of eight nucleotides, but those skilled in the art who have the benefit of this disclosure will recognize that the target-binding domains may vary in size from as few as three nucleotides up to as many as 25 or more nucleotides. In a preferred embodiment, the target-binding domains are comprised of seven to ten nucleotides such that the enzyme takes the form
5′-N₁-GGCTAGCTACAACGA-N₂-3′ [Seq. ID No. 7]
   where N₁ and N₂ represent any sequence of nucleotides that target a specific RNA ranging in size from 3 to 25 nucleotides, and preferably seven to ten nucleotides.

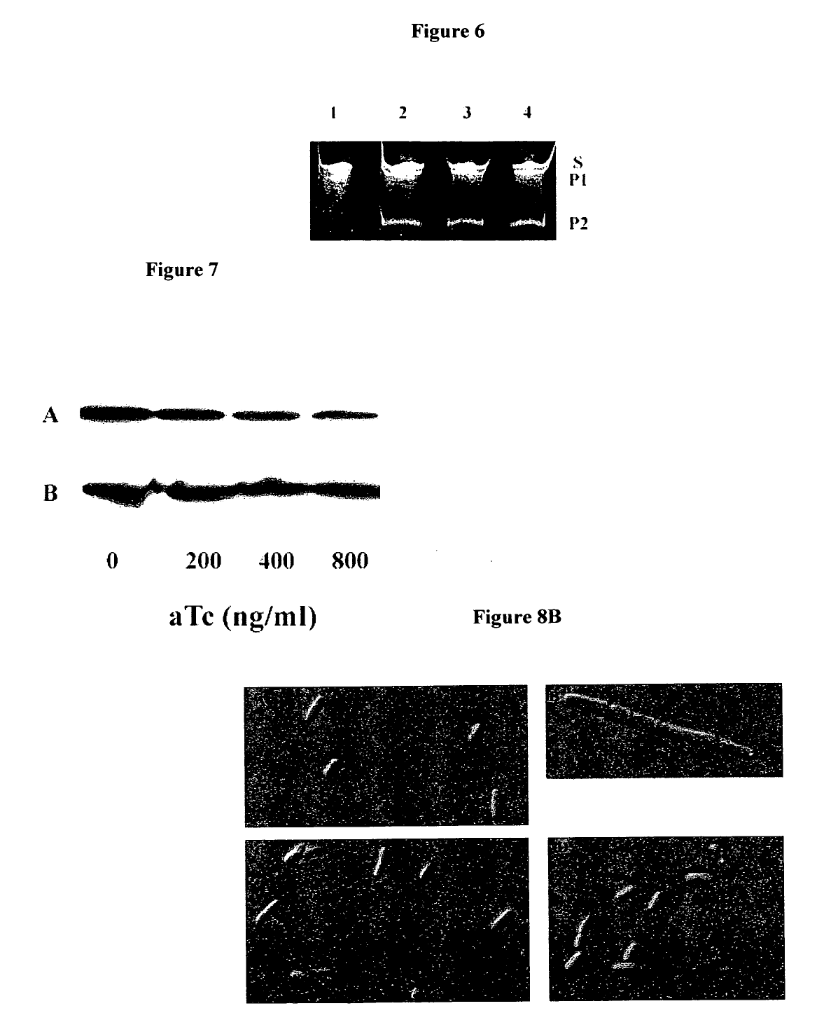

The ability of the expression vector to produce DNA enzyme molecules in bacterial cells may be tested by evaluating the cleavage activity of the designed DNA enzyme in a cell-free system. The 1225 nt ftsZ RNA, produced by in vitro transcription, may be used as a substrate and the cleavage assay may be carried out at 37° C. for various period of time as indicated in a 10 μl reaction containing 10 mM MgCl₂, 50 mM Tris-HCl, pH 7.5, 100 nM template ftsZ RNA, 100 μM DNA enzyme, and 10 units RNasin. As shown in FIG. 6, the synthetic DNA enzyme can effectively cleave the ftsZ RNA in the time as short as 0.5 hr, producing the products with expected sizes (368 nt and 857 nt).

Given that the designed DNA enzyme has been shown capable of cleaving ftsZ RNA in vitro (FIG. 6) and an aTc regulated expression vector can produce active RT (FIGS. 3 and 4), the expression vector may be used to generate the DNA enzyme in cells and the effects of the DNA enzyme on the expression of the ftsZ gene may then be determined. Bacterial cells carrying the pssXGb(FtsZ-DZ) vector (A) as well as negative control cells carrying the pssXGa vector (B) may be grown in the presence of various amount of aTc (0, 200, 400, 800 ng/ml) for 3 hrs. As shown in FIG. 7, compared to the cells grown in the absence of aTc, the FtsZ expression level in the bacterial cells carrying pssXGb(FtsZ-DZ) will be seen to be reduced significantly upon addition of aTc (FIG. 7A). This reduction is not observed in the control cells (FIG. 7B).

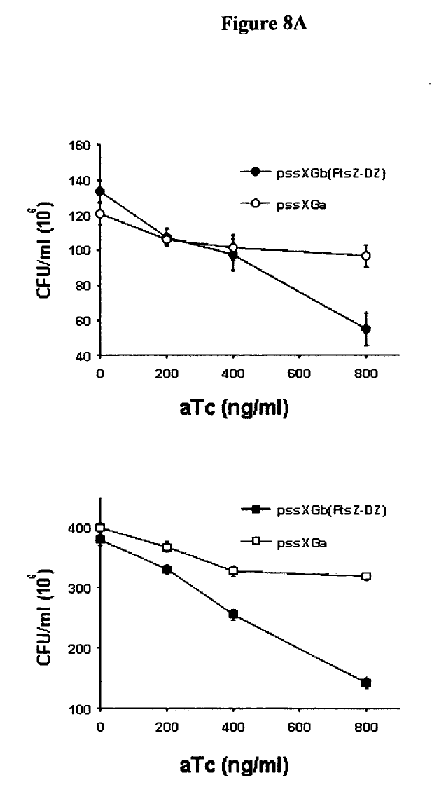

Because ftsZ gene is essential for bacterial division and viability and the result as shown in FIG. 7 indicates that the intracellularly-generated DNA enzyme significantly represses ftsZ expression, the effect of the expressed DNA on bacterial cell proliferation must be investigated. Bacterial cells carrying the pssXGb(FtsZ-DZ) vector grown in the presence of various amounts of aTc (0, 200, 400, 800 ng/ml) for either 1 or 2 hrs and viable cells may be enumerated. As shown in FIG. 8A, cell growth inhibition was in a time and aTc-concentration dependent manners. Furthermore, long filamentous cells were observed to be formed as a result of the cell growth inhibition (FIG. 8B).

Construction of a Tetracycline-Inducible ssDNA or ODN Expression Library.

The library inserts were generated by annealing three ODNs, CY(SacII)-40,

| | |
|---|---|
| CY(SacII)-40, | |
| CTCTCACTCC(N)40ACTGTTGAAAGGC | [Seq. ID No. 8] |
| CY(SacII)-L, | |
| CGGAGAGTGAGG | [Seq. ID No. 9] |
| and | |
| CY(SacII)-R, | |
| CTTTCAACAGT | [Seq. ID No. 10] |

at the molar ratio of 1:20:20. Here, “N” represents any of the bases A, T, C, or G. There are thus 40-mer sequences randomly synthesized and represented as CY(SacII)-40 ODNs. All the ODNs were mixed and denatured at 95° C. for 3 min and then cooled down slowly to room temperature over approximately 1 hr. Since CY(SacII)-L complements the left arm of CY(SacII)-40 while CY(SacII)-R complements the right arm of the same ODN, partial double-stranded ODNs are formed by the annealing process. The annealed ODN formed a partial double-stranded DNA and was filled in those remaining single-stranded Ns and blunt ended using the DNA Polymerase I, Large (Klenow) Fragment (New England Biolabs, Beverly, Mass.). The double-stranded DNA was then subcloned into newly created prokaryotic ssDNA expression vector designated pssXGb and subsequently transformed into bacterial cells, DH5αPRO using electroporation.

DNA enzyme expression library can be constructed similarly using a. DZlib1

[Seq. ID No. 11]

5′-CTCGAGTCTAGANNNNNNNNGGCTAGCTACAACGANNNNNNNNTTAA
TTAAGCTAGC-3′

(N can be either A, or T, or C or G) and b. DZlib2
5′-GCTAGCTTAATTAA-3′ [Seq. ID No. 12].
   These two oligonucleotides may be mixed at a molar ratio of 1:20(a:b). The two ODNs may be annealed together to form a partial double-stranded DNA by heating for 5 min at 75° C., then cooling slowly to room temperature. The recessed 3′ termini of the partial double-stranded DNA may be filled by Klenow fragment (NEB), and the resulting double-stranded DNA may be digested with PacI and XbaI. The double digested products can then be gel purified. After phenol extraction and ethanol precipitation, the PacI/XbaI digested library DNA may be cloned into the ssDNA expression vector, pssXGb.
   ssDNA Expression Library Screening.

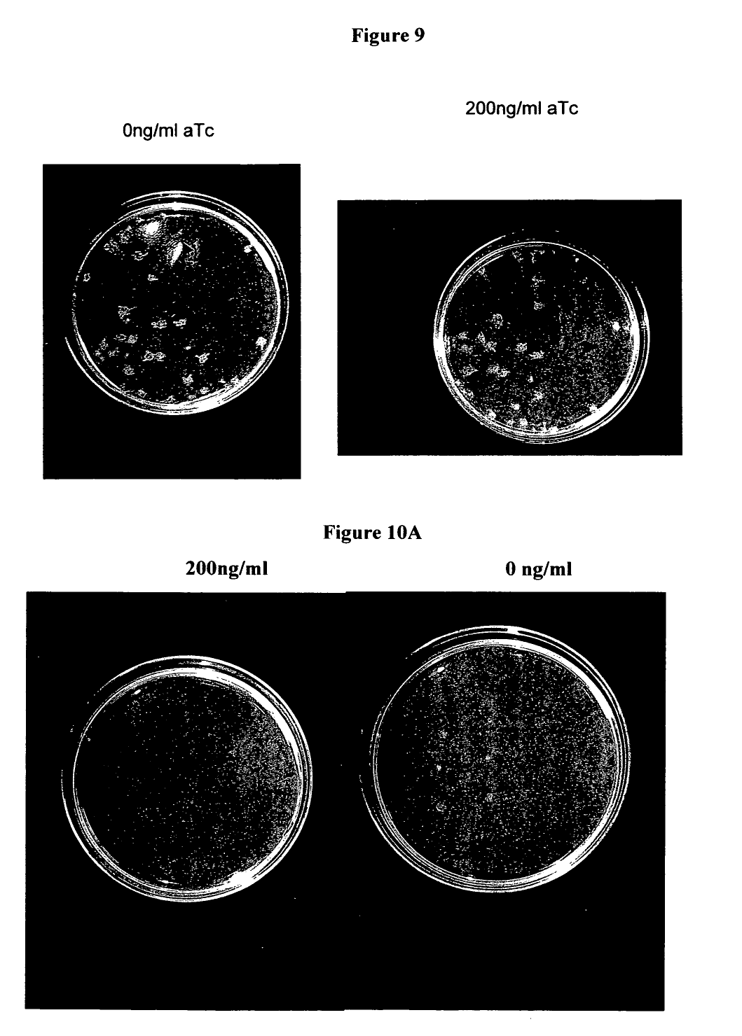

Since the ssDNA expression library was constructed based on a tetracycline inducible vector, bacterial cells containing the ssDNA expression library were plated in duplicate in Luria broth (LB) plates in the presence or absence 200 ng/ml aTc. Colonies growing only in the absence of aTc, as shown in FIG. 9, were identified as positive colonies. Plasmid DNA was extracted from the positive colony from the library screening, and the insert sequences may be determined by 3′ end single-pass sequencing. The insert generated by this set of experiments was named CYGX080103, with sequence of:

[Seq. ID No. 13]

5′-CTTTCAACAGTTTTGATGACCTTTGCTGACCATACAATTGCGATATC
GTGGGGAGTGAGAG-3′.

The sequence was then analyzed using GenBank BLASTN program to identify the potential gene targets based on sequence homologies. The ODNs comprised in the CYGX080103 and their potential genes that can be knocked down in accordance with the present invention include, but are not limited to, CYGX08010301
5′-CCTTTGCTGACCATAC-3′ [Seq. ID No. 14]
   and its target btuE (GenBank ID: NP-416225.1), CYGX08010302
5′-GACCTTTGCTGACCA-3′ [Seq. ID No. 15]
   and its target CaiB (GenBank ID: NP-414580.1), CYGX08010303
5′-ACAGTTTTGATGAC-3′ [Seq. ID No. 16]
   and its target ydgD (GenBank ID: NP-418152.1), CYGX08010304
5′-ACAATTGCGATAT-3′ [Seq. ID No. 17]
   and its target ygcQ (GenBank ID: NP-417249.2), CYGXO8010305
5′-GACCTTTGCTGAC-3′ [Seq. ID No. 18]
   and its target ftsh (GenBank ID: NP-417645.1), CYGX08010306
5′-TCAACAGTTTTGATGAC-3′ [Seq. ID No. 19]
   and its target ppiB (GenBank ID: NP-415058.1), CYGX08010307
5′-ATGACCTTTGCTG-3′ [Seq. ID No. 20]
   and its target yihl (GenBank ID: NP-418308.1), CYGX08010308
5′-CAGTTTTGATGA-3′ [Seq. ID No. 21]
   and its target zntA (GenBank ID: NP-417926.1), CYGX08010309
5′-ACCTTTGCTGAC-3′ [Seq. ID No. 22]
   and its target yicI (GenBank ID: NP-418116.1), CYGX08010310
5′-TTGCTGACCATA-3′ [Seq. ID No. 23]
   and its target fhuA (GenBank ID: NP-414692.1), CYGX08010311
5′-TGACCTTTGCTG-3′ [Seq. ID No. 24]
   and its target rplD (GenBank ID: NP-417778.1), CYGX08010312
5′-GTTTTGATGACC-3′ [Seq. ID No. 25]
   and its target ilvB (GenBank ID: NP-418127.1), CYGX08010313
5′-GCGATATCGTGG-3′ [Seq. ID No. 26]
   and its target lepB (GenBank ID: NP-417063.1), CYGX08010314
5′-TTGATGACCTTT-3′ [Seq. ID No. 27]
   and its target aroK (GenBank ID: NP-417849.1), CYGX08010315
5′-TGGGGAGTGAG-3′ [Seq. ID No. 28]
   and its target mfd (GenBank ID: NP-415632.1), CYGX08010316
5′-TTGCTGACCAT-3′ [Seq. ID No. 29]
   and its target rlpA (GenBank ID: NP-415166.1), CYGX08010317
5′-TTTTGATGACC-3′ [Seq. ID No. 30]
   and its target accA (GenBank ID: NP-414727.1), CYGX08010318
5′-TGATGACCTTT-3′ [Seq. ID No. 31]
   and its target pgpA (GenBank ID: NP-414952.1).

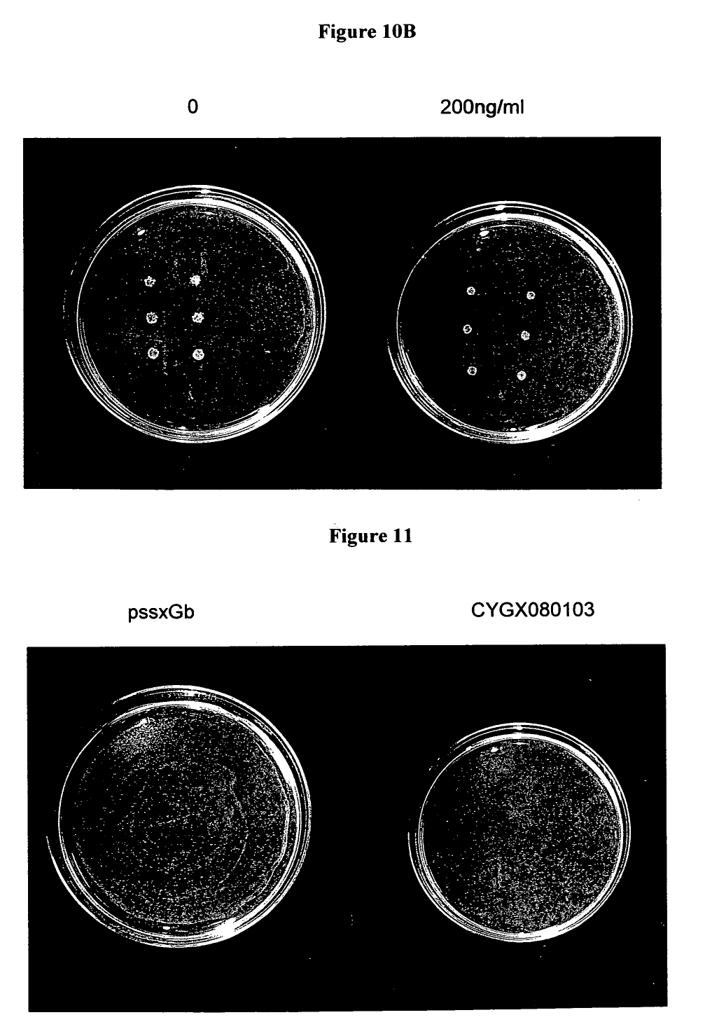

The antibacterial efficacy of the ODN CYGX080103 was further evaluated in two separate experiments. The first experiment was designed to examine the conditionally lethal effect of the ODN expression plasmid in the host cell DH5αPro, the bacterial strain employed in the library screening. The second experiment was designed to study the lethal effect of the ODN expression plasmid on other bacterial strains. In the first study, the ODN expression plasmid and the plasmid without ODN insert as negative control, may be transformed into DH5αPro, and plated onto LB media with or without 200 ng/ml aTc. As shown in FIG. 10A, DH5αPro cells carrying the ODN expression plasmid grow normally on the media without aTc, but not on the media with aTc. DH5αPro cells carrying the plasmid without the ODN insert grow on both media (FIG. 10B). In the second study, the ODN expression plasmid and the plasmid without ODN insert are observed to be transformed into E. coli XL10-gold(kan). The resulted transformants may be plated on LB media with chloramphenicol, and incubated at 37° C. O/N. As shown in FIG. 11, no XL10-gold(kan) carrying ODN expression plasmid grows on the LB media, while the XL10-gold(kan) carrying the plasmid without the ODN insert grows normally.

Those skilled in the art who have the benefit of this disclosure will recognize that certain changes can be made to the component parts of the apparatus of the present invention without changing the manner in which those parts function to achieve their intended result. All such changes, and others which will be clear to those skilled in the art from this description of the preferred embodiments of the invention, are intended to fall within the scope of the following, non-limiting claims.

1. An oligodeoxynucleotide (ODN) library comprising a plurality of oligodeoxynucleotides of specific length, at least one of the oligodeoxynucleotides comprising said ODN library being capable of interacting with a target genomic DNA, mRNA or protein when inserted into a DNA expression vector with the specific calling sequence for said oligodeoxynucleotide being embedded in said expression vector capable, said expression vector being capable of being introduced into a target cell to produce at least one of said oligodeoxynucleotides when induced by exposure to a chemical agent for interacting with genomic DNA, mRNA or protein with observable result.

2. A process for identifying and isolating an oligodeoxynucleotide comprising the steps of:
utilizing the ODN library of claim 1 to express a plurality of copies of at least one said oligodeoxynucleotide in the target cell;
growing the target cells into a colony of cells;
dividing the colony into paired colonies;
exposing one of the paired colonies to a chemical agent capable of inducing expression of said at least one oligodeoxynucleotide by the cells of the exposed colony, causing the expressed oligodoxynucleotide to interact with genomic DNA, mRNA or a protein to alter expression of a gene;
observing the result in said exposed cells; and
sequencing the DNA of the cells of the unexposed colony to identify the sequence of the library oligodeoxynucelotide that caused alteration of the gene.

3. The method of claim 2 wherein said cells are bacteria strain DH5αPro.

4. The plasmid pssXG.

5. The plasmid of claim 4 comprising a PBS having the sequence 5′-TGGTGCGTCCGAG-3′ [Seq. ID No. 3].

6. A cell having the plasmid of claim 4 transformed therein.

7. A prokaryotic cell having the plasmid of claim 4 transformed therein.

8. The plasmid of claim 4 comprising a sequence coding for in vivo expression of a single-stranded DNA enzyme targeted to the bacterial FtsZ gene.

9. The plasmid of claim 8 wherein the single-stranded DNA enzyme is specific for a GU site at position 880 of the bacterial FtsZ gene.

10. The plasmid of claim 8 wherein the single-stranded DNA enzyme comprises 5′-N₁-GGCTAGCTACAACGA-N₂-3′ [Seq. ID No. 7] where N₁ and N₂ represent any sequence of nucleotides ranging in size from about seven to about ten nucleotides that target a specific RNA.

11. A cell having the plasmid of claim 8 transformed therein.

12. A single-stranded DNA enzyme comprising a 15 nucleotide catalytic domain flanked by random RNA target-binding domains of between about 7 and about 10 nucleotides each.

13. The single-stranded DNA enzyme of claim 12 wherein said catalytic domain comprises the sequence 5′-N₁-GGCTAGCTACAACGA-N₂-3′ [Seq. ID No. 7], where N₁ nd N₂ represent any sequence of nucleotides ranging in size from about seven to about ten nucleotides that target a specific RNA.

14. A plasmid having the DNA enzyme of claim 12 contained therein.

15. A cell having the plasmid of claim 14 transformed therein.


SEQUENCE LISTING (33 sequences)

SEQ ID NO: 1 - DNA, 36 bp, Artificial Sequence
  Description of Artificial Sequence Synthetic oligonucleotide
ctagctagctagcgatcgatgggaccaatggggcag

SEQ ID NO: 2 - DNA, 23 bp, Artificial Sequence
  Description of Artificial Sequence Synthetic oligonucleotide
cggggtaccagtattccctggtc

SEQ ID NO: 3 - DNA, 13 bp, Artificial Sequence
  Description of Artificial Sequence Synthetic oligonucleotide
tggtgcgtccgag

SEQ ID NO: 4 - DNA, 76 bp, Artificial Sequence
  Description of Artificial Sequence Synthetic oligonucleotide
taactggatgatcgttgtagctagccttcgaaacttggtggtgcgtccgagtggaccgggagacccctgctcgagt

SEQ ID NO: 5 - DNA, 82 bp, Artificial Sequence
  Description of Artificial Sequence Synthetic oligonucleotide
ctagactcgagcaggggtctcccggtccactcggacgcaccaccaagtttcgaaggctagctacaacgatcatccagttaat

SEQ ID NO: 6 - DNA, 31 bp, Artificial Sequence
  Description of Artificial Sequence Synthetic oligonucleotide
gtttcgaaggctagctacaacgatcatccag

SEQ ID NO: 7 - DNA, 65 bp, Artificial Sequence
  Description of Artificial Sequence Synthetic oligonucleotide
nnnnnnnnnnnnnnnnnnnnnnnnnggctagctacaacgannnnnnnnnnnnnnnnnnnnnnnnn

SEQ ID NO: 8 - DNA, 63 bp, Artificial Sequence
  Description of Artificial Sequence Synthetic oligonucleotide
ctctcactccnnnnnnnnnnnnnnnnnnnnnnnnnnnnnnnnnnnnnnnnactgttgaaaggc

SEQ ID NO: 9 - DNA, 12 bp, Artificial Sequence
  Description of Artificial Sequence Synthetic oligonucleotide
cggagagtgagg

SEQ ID NO: 10 - DNA, 11 bp, Artificial Sequence
  Description of Artificial Sequence Synthetic oligonucleotide
ctttcaacagt

SEQ ID NO: 11 - DNA, 57 bp, Artificial Sequence
  Description of Artificial Sequence Synthetic oligonucleotide
ctcgagtctagannnnnnnnggctagctacaacgannnnnnnnttaattaagctagc

SEQ ID NO: 12 - DNA, 14 bp, Artificial Sequence
  Description of Artificial Sequence Synthetic oligonucleotide
gctagcttaattaa

SEQ ID NO: 13 - DNA, 61 bp, Artificial Sequence
  Description of Artificial Sequence Synthetic oligonucleotide
ctttcaacagttttgatgacctttgctgaccatacaattgcgatatcgtggggagtgagag

SEQ ID NO: 14 - DNA, 16 bp, Artificial Sequence
  Description of Artificial Sequence Synthetic oligonucleotide
cctttgctgaccatac

SEQ ID NO: 15 - DNA, 15 bp, Artificial Sequence
  Description of Artificial Sequence Synthetic oligonucleotide
gacctttgctgacca

SEQ ID NO: 16 - DNA, 14 bp, Artificial Sequence
  Description of Artificial Sequence Synthetic oligonucleotide
acagttttgatgac

SEQ ID NO: 17 - DNA, 13 bp, Artificial Sequence
  Description of Artificial Sequence Synthetic oligonucleotide
acaattgcgatat

SEQ ID NO: 18 - DNA, 13 bp, Artificial Sequence
  Description of Artificial Sequence Synthetic oligonucleotide
gacctttgctgac

SEQ ID NO: 19 - DNA, 17 bp, Artificial Sequence
  Description of Artificial Sequence Synthetic oligonucleotide
tcaacagttttgatgac

SEQ ID NO: 20 - DNA, 13 bp, Artificial Sequence
  Description of Artificial Sequence Synthetic oligonucleotide
atgacctttgctg

SEQ ID NO: 21 - DNA, 12 bp, Artificial Sequence
  Description of Artificial Sequence Synthetic oligonucleotide
cagttttgatga

SEQ ID NO: 22 - DNA, 12 bp, Artificial Sequence
  Description of Artificial Sequence Synthetic oligonucleotide
acctttgctgac

SEQ ID NO: 23 - DNA, 12 bp, Artificial Sequence
  Description of Artificial Sequence Synthetic oligonucleotide
ttgctgaccata

SEQ ID NO: 24 - DNA, 12 bp, Artificial Sequence
  Description of Artificial Sequence Synthetic oligonucleotide
tgacctttgctg

SEQ ID NO: 25 - DNA, 12 bp, Artificial Sequence
  Description of Artificial Sequence Synthetic oligonucleotide
gttttgatgacc

SEQ ID NO: 26 - DNA, 12 bp, Artificial Sequence
  Description of Artificial Sequence Synthetic oligonucleotide
gcgatatcgtgg

SEQ ID NO: 27 - DNA, 12 bp, Artificial Sequence
  Description of Artificial Sequence Synthetic oligonucleotide
ttgatgaccttt

SEQ ID NO: 28 - DNA, 11 bp, Artificial Sequence
  Description of Artificial Sequence Synthetic oligonucleotide
tggggagtgag

SEQ ID NO: 29 - DNA, 11 bp, Artificial Sequence
  Description of Artificial Sequence Synthetic oligonucleotide
ttgctgaccat

SEQ ID NO: 30 - DNA, 11 bp, Artificial Sequence
  Description of Artificial Sequence Synthetic oligonucleotide
ttttgatgacc

SEQ ID NO: 31 - DNA, 11 bp, Artificial Sequence
  Description of Artificial Sequence Synthetic oligonucleotide
tgatgaccttt

SEQ ID NO: 32 - RNA, 24 nt, Artificial Sequence
  Description of Artificial Sequence Synthetic oligonucleotide
cgucuggaugaguucgaaacggua

SEQ ID NO: 33 - protein, 12 aa, Artificial Sequence
  Description of Artificial Sequence Synthetic 6xHN tag
HNHNHNHNHNHN